(19) United States
(12) Patent Application Publication
(10) Pub. No.: US 2023/0065363 A1
(43) Pub. Date: Mar. 2, 2023

(54) SYSTEM FOR RECIRCULATING WARM AIR

(71) Applicant: Serengeti Warming Systems, LLC, Temple, GA (US)

(72) Inventor: Andrew Floyd Malen, Temple, GA (US)

(21) Appl. No.: 17/969,767

(22) Filed: Oct. 20, 2022

Related U.S. Application Data
(63) Continuation-in-part of application No. 17/412,373, filed on Aug. 26, 2021.

(51) Int. Cl. A47G 9/02; A47C 27/08; A61L 2/14; A61L 9/22

(52) U.S. Cl. CPC A47G 9/0215; A47C 27/082; A61L 2/14; A61L 9/22; A47G 2009/003

(57) ABSTRACT

A recirculating warm air system configured to connect to an air inflatable product is provided. The system includes a first chamber and a second chamber. The first chamber includes a first air inlet and a first valve located at the first air inlet. The second chamber includes a second valve located between the first chamber and the second chamber, an air outlet, and a first hose connected to the air outlet. The second chamber further includes an air blower and a heater located between the air blower and the air outlet. The second chamber includes a second air inlet, a third valve located at the second air inlet, and a second hose connected to the second air inlet. The first hose and the second hose are configured to connect to the air inflatable product. The system further includes a processing unit configured to control elements of the system.

### RELATED APPLICATION

This application is a Continuation-In-Part of U.S. application Ser. No. 17/412,373 filed on Aug. 26, 2021, which is incorporated herein by reference.

It is intended that the referenced application may be applicable to the concepts and embodiments disclosed herein, even if such concepts and embodiments are disclosed in the referenced applications with different limitations and configurations and described using different examples and terminology.

### TECHNICAL FIELD

The present invention relates generally to systems for recirculating warm air and, more particularly, to a recirculating warm air machine, an air inflatable blanket, and a cleaning unit for decontaminating the recirculating warm air machine.

### BACKGROUND

Thermostabilizing products such as blankets and mattresses are widely used in medicine to keep patients warm. A conventional thermostabilizing blanket is made of porous materials connected to a warm air circulating machine providing warm air to the thermostabilizing blanket through a hose. The thermostabilizing blanket is inflated by the warm air received from the warm air circulating machine.

Because the thermostabilizing blanket is porous, the warm air passes from the inside of the thermostabilizing blanket to the environment. The warm air circulating machine then uses the air from the environment to continue inflating the thermostabilizing blanket. However, the air in the environment can be contaminated during medical procedures. Therefore, the warm air circulating machine may circulate the contaminated air from the environment through the thermostabilizing blanket. Because the same warm air circulating machine with the same hose is usually used for multiple patients, the contaminated air obtained from the room environment of one patient can be released into the room environment of another patient.

Moreover, continuous air flow from the thermostabilizing blanket to an area where a surgery is performed may be problematic, especially when the air exiting the thermostabilizing blanket is contaminated.

An additional concern is contamination of the inner workings of the recirculating warm air machine. Contaminants from the air may aggregate and settle within the recirculating warm air machine, contaminating the air that is passed into the thermostabilizing blanket.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

Embodiments of the present disclosure may include a recirculating warm air system that comprises an air inflatable product made of an air-impermeable material. Embodiments may also include a recirculating warm air machine configured to supply air to the air inflatable product when removably attached thereto, the machine including a first chamber having a first air inlet configured to allow air to pass into the first chamber.

Embodiments may also include a second chamber having a second air inlet configured to allow air to pass into the second chamber and an air outlet configured to allow air to pass out of the second chamber. Embodiments may also include a plurality of one-way valves that may be configured to selectively allow the passage of air the first and second chambers to ensure re-circulation of heated air within the air inflatable product when the inflatable product is attached to the recirculating warm air machine.

Embodiments may also include a detachable cleaning unit configured to removably attach to the recirculating warm air machine, the cleaning unit including an ionizing unit, an intake hose configured to connect to the air outlet of the recirculating warm air machine, and an outlet hose configured to connect to either the first air inlet or the second air inlet of the recirculating warm air machine. When the cleaning unit is attached to the recirculating warm air machine, air may be configured to pass from the second chamber of the recirculating warm air machine into the ionizing unit via the intake hose, through the ionizing unit, and back into the recirculating warm air machine via the outlet hose.

In some embodiments, the recirculating warm air machine further comprises a first hose configured to connect to the air outlet and to the air inflatable product to pass the air from the recirculating warm air machine to the air inflatable product and a second hose configured to connect to the second air inlet and to the air inflatable product to receive the air passing from the air inflatable product.

In some embodiments, the first and/or second air inlets have one-way valves located thereon, the one-way valves configured to only allow air to flow into the recirculating warm air machine. In still further embodiments, the air outlet may have a one-way valve located thereon that is configured to only allow air to flow out of the recirculating warm air machine.

In some embodiments, the recirculating warm air machine further comprises an air blower configured to move air toward the air outlet and a heater located between the air blower and the air outlet. In still further embodiments, the recirculating warm air machine further comprises a processing unit configured to control one or more of the cleaning unit, the air blower, and the heater. Where the processing unit is configured to control the cleaning unit, it may be configured to control a time period for which the cleaning unit runs. In some embodiments, the processing unit may be configured to determine that the cleaning unit is connected to the recirculating warm air machine. In some embodiments, the processing unit may be configured to determine that the air inflatable product is connected to the recirculating warm air machine. A valve located on the first air inlet may be opened upon determining that the air inflatable product is connected to the recirculating warm air machine.

In some embodiments, the processing unit is further configured to determine, based on one or more criteria, that an amount of the air provided to the air inflatable product has reached a predetermined threshold and, based on that determination, close a second valve located between the first air chamber and the second air chamber, and open a third valve located at the second air inlet. Upon closing of the second valve and opening of the third valve, the air may recirculate through the air inflatable product and the second chamber.

Embodiments of the present disclosure may include a recirculating warm air system, the system including a recirculating warm air machine configured to supply air to an air inflatable product when removably attached thereto, the machine including a first chamber having a first air inlet configured to allow air to pass into the first chamber. Embodiments of the recirculating air machine may also include a second chamber having a second air inlet configured to allow air to pass into the second chamber and an air outlet configured to allow air to pass out of the second chamber.

Embodiments of the recirculating warm air machine may also include a plurality of one-way valves that may be configured to selectively allow the passage of air between the first and second chambers to ensure re-circulation of heated air within the air inflatable product when the inflatable product is attached to the recirculating warm air machine. Embodiments may also include a cleaning unit permanently attached to the recirculating warm air machine. The cleaning unit may include an ionizing unit and a hose in operative communication with the second chamber, the hose being configured to deliver negative ions into the second chamber.

In some embodiments, the warm air recirculating machine comprises an air blower configured to move air toward the air outlet and a heater located between the air blower and the air outlet. In still further embodiments, the recirculating warm air system comprises a processing unit configured to control one or more of the ionizing unit, the air blower, and the heater. The processing unit may be configured to control, as a non-limiting example, a time period for which the ionizing unit runs. In still further embodiments, a user may be able to select a timer period for which the ionizing unit runs.

In some embodiments, the processing unit may be configured to determine that the air inflatable product is connected to the warm air recirculating machine.

Embodiments of the present disclosure may include a method of cleaning the recirculating warm air system. The method may include attaching the intake hose of the cleaning unit to the air outlet of the recirculating warm air machine. Embodiments may also include attaching the outlet hose of the cleaning unit to the first air inlet and/or the second air inlet of the recirculating warm air machine. Embodiments may include setting a time period for which the cleaning unit runs. In some embodiments, the method may include, prior to attaching the intake and outlet hoses of the cleaning unit, detaching the air inflatable product from recirculating warm air machine.

Additional objects, advantages, and novel features will be set forth in part in the detailed description section of this disclosure, which follows, and in part will become apparent to those skilled in the art upon examination of this specification and the accompanying drawings or may be learned by production or operation of the example embodiments. The objects and advantages of the concepts may be realized and attained by means of the methodologies, instrumentalities, and combinations particularly pointed out in the appended claims.

### BRIEF DESCRIPTION OF THE DRAWINGS

The features of the invention are set forth with particularity in the appended claims. A better understanding of the features and advantages of the present invention will be obtained by reference to the following detailed description that sets forth illustrative embodiments, in which the principles of the invention are utilized, and which the accompanying drawings illustrate.

FIG. 1 is a schematic diagram of a recirculating warm air system, according to an example embodiment;

FIG. 2 shows modules of a recirculating warm air system, according to an example embodiment;

FIG. 3A shows a schematic diagram of using air inflatable products with a recirculating warm air machine, according to an example embodiment;

FIG. 3B shows a schematic diagram of using air inflatable products with a recirculating warm air machine, according to an example embodiment;

FIG. 4 shows a schematic diagram of a recirculating warm air system, according to an example embodiment;

FIG. 5 shows a schematic diagram of a recirculating warm air system, according to an example embodiment;

FIG. 6 is a computing system that can be used to implement a recirculating warm air system, according to an example embodiment; and

FIG. 7 is a flow chart illustrating a method of using a recirculating warm air system, according to an example embodiment.

### DETAILED DESCRIPTION

The following detailed description includes references to the accompanying drawings, which form a part of the detailed description. The drawings show illustrations in accordance with exemplary embodiments. These exemplary embodiments, which are also referred to herein as “examples,” are described in enough detail to enable those skilled in the art to practice the present subject matter. The embodiments can be combined, other embodiments can be utilized, or structural, logical, and electrical changes can be made without departing from the scope of what is claimed. The following detailed description is, therefore, not to be taken in a limiting sense, and the scope is defined by the appended claims and their equivalents.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Regarding applicability of 35 U.S.C. § 112, ¶6, no claim element is intended to be read in accordance with this statutory provision unless the explicit phrase “means for” or “step for” is actually used in such claim element, whereupon this statutory provision is intended to apply in the interpretation of such claim element.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header.

The present disclosure includes many aspects and features. Moreover, while many aspects and features relate to, and are described in, the context of heated blankets in a medical setting, embodiments of the present disclosure are not limited to use only in this context.

The present disclosure is directed to a system for recirculating warm air. The system may comprise, a recirculating warm air machine, an air inflatable product, such as a blanket or a mattress, and/or a cleaning unit for decontaminating the recirculating warm air machine. In some embodiments, the air inflatable product is made of non-porous air-impermeable material. The recirculating warm air machine may be connected to the air inflatable product and/or the cleaning unit via one or more hoses.

The recirculating warm air machine may include two chambers. The first chamber may include a first air inlet and a first valve configured to pass air into the first chamber. The second chamber may include a second valve located between the first chamber and the second chamber. The second valve may pass the air from the first chamber to the second chamber. In some embodiments, the second chamber may include an air outlet, an air blower, and a heater located between the air blower and the air outlet.

In example embodiments, when the recirculating warm air machine is turned on, the air blower starts moving the air from the first air inlet to the first chamber to the second valve located between the first chamber and the second chamber. Upon entering the second chamber, the air may pass through the air blower and the heater. The heater may heat the air passing through the heater. In such embodiments, after the air is heated, the air moves to the air outlet in the second chamber, passes through a first hose to the air inflatable product, and inflates the air inflatable product.

In example embodiments, when the pressure in the air inflatable product reaches a predetermined threshold, a third valve located in the second chamber may open and the air may pass through the second hose from the air inflatable product to the second chamber.

In example embodiments, once the third valve opens, the second valve between the first chamber and the second chamber may close. In such embodiments, after the second valve closes, the heated air may be recirculated through the air inflatable product and the second chamber.

Thus, the recirculating warm air system recirculates the air in a closed loop without releasing the air into the environment. Accordingly, the air recirculated inside the recirculating warm air system and the air inflatable product does not come in contact with the environment and cannot be contaminated by bacteria residing in the environment.

Moreover, because the air inflatable product is non-porous, no air is released by the air inflatable product into the environment. Therefore, there is no air flowing from the air inflatable product into a surgery area during medical procedures.

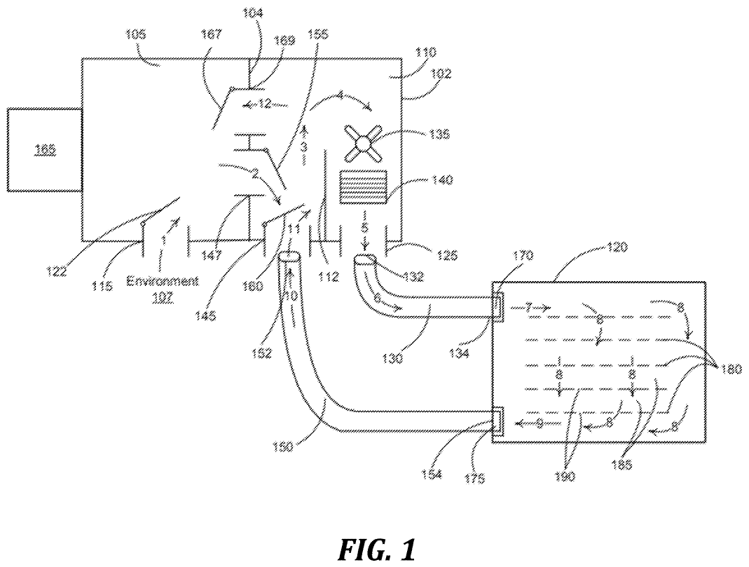

Referring now to the drawings, FIG. 1 is a schematic diagram of a recirculating warm air system 100 (also referred to as a system 100), according to an example embodiment. The system 100 may include a recirculating warm air machine 102 configured to connect to an air inflatable product 120. In an example embodiment, the system may include both the recirculating warm air machine 102 and the air inflatable product 120. The recirculating warm air machine 102 may be configured to inflate the air inflatable product 120 with warm air and recirculate the warm air through the air inflatable product 120.

In example embodiments, the air inflatable product 120 may include one of the following: a blanket, a mattress, a bag, a suit, and any other product to be inflated with air. The air inflatable product 120 may be used in medicine to keep patients warm. For example, patients may be laid onto an air inflatable mattress, covered by an air inflatable blanket, laid into an air inflatable bag, or clothed in an air inflatable suit during a medical procedure, such as a medical surgery.

The system 100 may include a first chamber 105 and a second chamber 110 of the recirculating warm air machine 102. The system 100 may further include a chamber separator 104 disposed between the first chamber 105 and the second chamber 110 to separate the first chamber 105 and the second chamber 110 from each other. The chamber separator 104 may include a wall made inside the recirculating warm air machine 102. In some example embodiments, the first chamber 105 and the second chamber 110 may be of the same or different sizes.

The first chamber 105 may include a first air inlet 115 and a first valve 122 located at the first air inlet 115. The first valve 122 may pass air from an environment 107 into the first chamber 105. In an example embodiment, the first air inlet 115 may have the size of about 2-3 inches.

The second chamber 110 may further include a second valve 155 located between the first chamber 105 and the second chamber 110. The second valve 155 may be disposed in an opening 147 made in the chamber separator 104. The second valve 155 may pass the air from the first chamber 105 to the second chamber 110.

The second chamber 110 may have an air outlet 125 and a second air inlet 145. In an example embodiment, the second air inlet 145 and the air outlet 125 may have the size of about 2-3 inches. The second chamber 110 may further include an air blower 135 configured to move the air towards the air outlet 125. The second chamber 110 may have a heater 140 located between the air blower 135 and the air outlet 125.

The second chamber 110 may be attached to a first hose 130 and a second hose 150. The first hose 130 and the second hose 150 may be made of a flexible air-impermeable material, such as plastic or any other applicable material.

The air inflatable product 120 may connect to the second chamber 110 using the first hose 130 and the second hose 150. Specifically, the first hose 130 may connect to the air outlet 125 by a first end 132 of the first hose 130 and may connect to the air inflatable product 120 by a second end 134 of the first hose 130. When connected, the first hose 130 may pass the air from the second chamber 110 to the air inflatable product 120.

The second chamber 110 may further include a second air inlet 145. The second hose 150 may be connected to the second air inlet 145. The second hose 150 may connect to the air inflatable product 120 and receive the air from the air inflatable product 120. Specifically, the second hose 150 may include a first end 152 and a second end 154. The first end 152 may connect to the second air inlet 145 and the second end 154 may connect to the air inflatable product 120. When connected, the second hose 150 may pass the air from the air inflatable product 120 to the second chamber 110.

The second chamber 110 may further include a third valve 160 located at the second air inlet 145. The third valve 160 may be configured to pass the air received from the air inflatable product 120 into the second chamber 110.

The system 100 may further include a processing unit 165. The processing unit 165 may be configured to control an operation of elements of the system 100. Specifically, the processing unit 165 may be configured to control one or more of the air blower 135, the heater 140, the first valve 122, the second valve 155, the third valve 160, and a fourth valve 167.

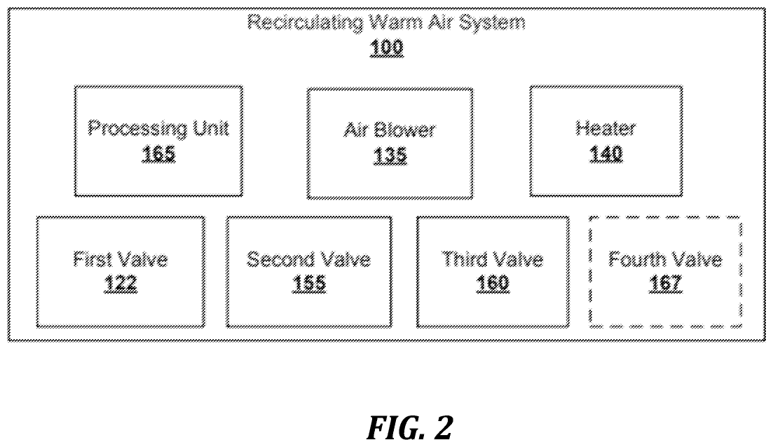

FIG. 2 shows modules of the recirculating warm air system 100, according to an example embodiment. The system 100 may include a processing unit 165, an air blower 135, a heater 140, a first valve 122, a second valve 155, a third valve 160, and, optionally, a fourth valve 167. The processing unit 165 may be configured to issue control commands to control the operation of any of the air blower 135, the heater 140, the first valve 122, the second valve 155, the third valve 160, and the fourth valve 167.

Referring again to FIG. 1, upon turning the system 100 on, the processing unit 165 may issue control commands to open the first valve 122 and the second valve 155 and close the third valve 160. Then, the processing unit 165 may issue control commands to turn the air blower 135 and the heater 140 on. In example embodiments, turning the air blower 135 on initiates an intake of the air through the first valve 122 from an environment 107 into the first chamber 105, as shown by arrow 1 in FIG. 1. The air may then be moved through the second valve 155 from the first chamber 105 to the second chamber 110, as shown by arrow 2.

In the second chamber 110, the air may be moved from the second valve 155 to the air blower 135 (as shown by arrow 3) and then through the air blower 135 and the heater 140 (as shown by arrow 4). The heater 140 may be configured to heat the air passing from the air blower 135 to the air outlet 125 to a predetermined temperature. In an example embodiment, the second chamber 110 may further include a guide wall 112. The guide wall 112 may be disposed in the second chamber 110 in order to direct the air from the second valve 155 to the air blower 135.

Upon passing through or in a proximity of the heater 140, the air may pass to the air outlet 125 (as shown by arrow 5). Thus, in such embodiments, the air is passed from the second chamber 110 to the air inflatable product 120. Specifically, the air is passed from the air outlet 125 of the second chamber 110 to the first hose 130 and then through the first hose 130 (as shown by arrow 6) to the air inflatable product 120. The third valve 160, when closed, may prevent the air from exiting the air inflatable product 120. Therefore, as the air blower 135 continues to move the air, the air may inflate the air inflatable product 120 (as shown by arrow 7).

The air inflatable product 120 may be made of a flexible non-porous air-impermeable material, such as plastic, rubber, insulated cloth, and/or the like. In an example embodiment, the air inflatable product 120 may be in a shape of a parallelepiped with rounded corners and edges. Any other applicable shapes of the air inflatable product 120 can be used for specific purposes.

The air inflatable product 120 may have a first inlet 170 configured to attach to the first hose 130 and a second inlet 175 configured to attach to the second hose 150. The air may pass to the inflatable product 120 through the first inlet 170 (as shown by arrow 7).

The air inflatable product 120 may have one or more dividers 180. The dividers 180 may connect an upper surface and a lower surface of the air inflatable product 120 and divide the air inflatable product 120 into sections 185. The dividers 180 may have air passages 190 for passing the air between sections 185. The air provided into the air inflatable product 120 may inflate the air inflatable product 120 by moving along the sections 185 and through the air passages 190 (as shown by arrow 8).

The processing unit 165 may be configured to determine, based on predetermined criteria, that the amount of the air provided to the air inflatable product has reached a predetermined threshold. For example, the third valve 160 may be set to maintain a predetermined pressure in the air inflatable product 120. Therefore, the third valve 160 may be closed until the pressure in the air inflatable product 120 reaches the predetermined pressure. Reaching the predetermined pressure in the air inflatable product 120 means that the amount of the air provided to the air inflatable product 120 has reached the predetermined threshold. The predetermined pressure maintained by the third valve 160 may be a full inflation pressure of the air inflatable product 120 such that the air inflatable product 120 remains full of warm air.

Upon determining that the amount of the air provided to the air inflatable product 120 has reached the predetermined threshold, the processing unit 165 may close the second valve 155 and open the third valve 160. In other example embodiments, opening of the third valve 160 increases pressure in the second chamber 110, thus closing the second valve 155 between the first chamber 105 and the second chamber 110 and closing the first valve 122 where air enters the recirculating warm air machine 102. Thus, upon closing of the second valve 155 and the opening of the third valve 160, a closed warm air circuit is created in which the air recirculates through the air inflatable product 120 and the second chamber 110. Specifically, the air exits from the second inlet 175 of the air inflatable product 120 (as shown by arrow 9), passes through the second hose 150 (as shown by arrow 10), and enters the second chamber 110 through the second air inlet 145 (as shown by arrow 11). Upon entering the second chamber 110, the air again travels to the air blower 135. The air flow from the air blower 135 and the pressure in the air inflatable product 120 keep the third valve 160 open and keep circulation of the warm air continuous until the system 100 is turned off. Accordingly, the system 100 recirculates the air through the air inflatable product 120 and the second chamber 110 without releasing the air into the environment 107.

In an example embodiment, the system 100 may further include a fourth valve 167 located between the first chamber 105 and the second chamber 110. The fourth valve 167 may be disposed in an opening 169 made in the chamber separator 104. The fourth valve 167 may include a pressure pop-off valve configured to pass the air from the second chamber 110 to the first chamber 105 (as shown by arrow 12), if pressure in the air inflatable product 120 exceeds a predetermined pressure (i.e., if excess pressure is applied to the air inflatable product 120). Upon releasing the air from the second chamber 110 to the first chamber 105, the fourth valve 167 may close again when the pressure returns to the predetermined level in the air inflatable product 120. In such embodiments, when the fourth valve 167 opens, the first valve 122 in the first chamber 105 is already closed, thus no air is released from the first chamber 105 into the environment 107.

In an example embodiment, each of the first valve 122, the second valve 155, the third valve 160, and the fourth valve 167 may be one-way valves that allow an air flow to pass in one direction and are completely closed in the opposite direction. Specifically, the first valve 122 may allow the air flow to pass only in the direction from the environment 107 to the first chamber 105. The second valve 155 may allow the air flow to pass only in the direction from the first chamber 105 to the second chamber 110. The third valve 160 may allow the air flow to pass only in the direction from the second hose 150 to the second chamber 110. The fourth valve 167 may allow the air flow to pass only in the direction from the second chamber 110 to the first chamber 105.

In some embodiments, the processing unit 165 may be configured to determine that the first hose 130 and/or the second hose 150 are connected to the second chamber 110. To prevent release of the air from the second chamber 110 into the environment 107, the opening of the first valve 122 may be initiated upon determining that the first hose 130 and the second hose 150 are connected to the second chamber 110. The air outlet 125 and the second air inlet 145 may include sensors to determine whether the first hose 130 is connected to the air outlet 125 and whether the second hose 150 is connected to the second air inlet 145. Thus, the system 100 has a built-in safety feature where the first hose 130 and the second hose 150 must be connected for the recirculating warm air machine 102 to turn on, thus forbidding the recirculating warm air machine 102 to free the air into the environment 107 instead of the air inflatable product 120.

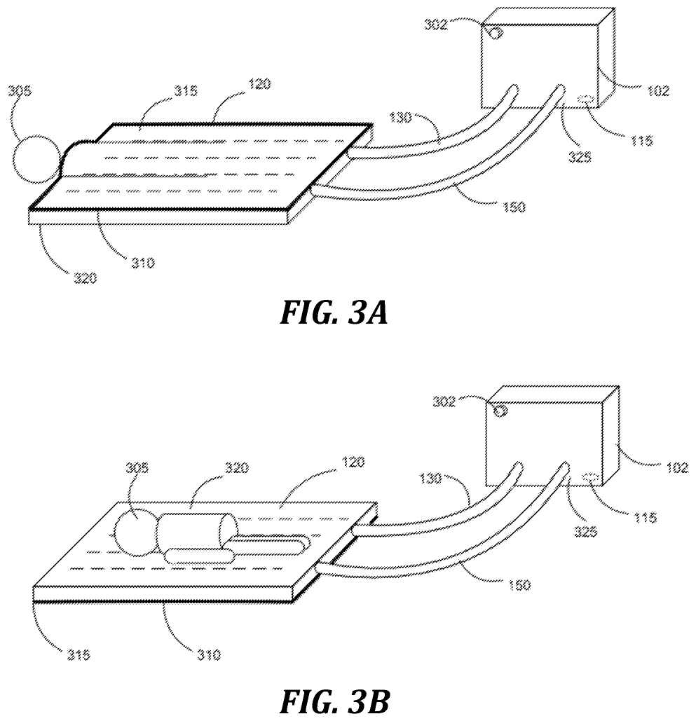

FIGS. 3A and 3B show schematic diagrams 300 and 350, respectively, of using air inflatable products with a recirculating warm air machine, according to an example embodiment. The recirculating warm air machine 102 may have an on/off switch 302 to switch the recirculating warm air machine 102 on and off. The air inflatable product 120 may be connected to the recirculating warm air machine 102 using a first hose 130 and a second hose 150. The first air inlet 115 may be located on a bottom 325 of the recirculating warm air machine 102.

FIG. 3A shows an example embodiment, in which the air inflatable product 120 is a blanket. A patient 305 may be covered with the air inflatable product 120. The recirculating warm air machine 102 may recirculate the warm air through the air inflatable product 120 to keep the patient 305 warm.

In an example embodiment, the air inflatable product 120 may include a heat reflective backing 310. Specifically, the air inflatable product 120 may have a first surface 315 and a second surface 320. The second surface 320 may be intended to face the patient 305. The heat reflective backing 310 may be applied to the first surface 315 and configured to reflect the heat from an inner side of the first surface 315 down towards the second surface 320. The second surface 320 may not have any heat reflective backing. Therefore, the heat from the second surface 320 may contact the patient 305 through the second surface 320 to keep the patient 305 warm.

FIG. 3B shows an example embodiment, in which the air inflatable product 120 is a mattress. A patient 305 may be laid onto the air inflatable product 120. The recirculating warm air machine 102 may recirculate the warm air through the air inflatable product 120 to keep the patient 305 warm. The air inflatable product 120 may include a heat reflective backing 310. Specifically, the air inflatable product 120 may have a first surface 315 and a second surface 320. The second surface 320 may be intended to face the patient 305. The heat reflective backing 310 may be applied to the first surface 315 and configured to reflect the heat from an inner side of the first surface 315 up towards the second surface 320. The second surface 320 may not have any heat reflective backing. Therefore, the heat from the second surface 320 may contact the patient 305 through the second surface 320 to keep the patient 305 warm.

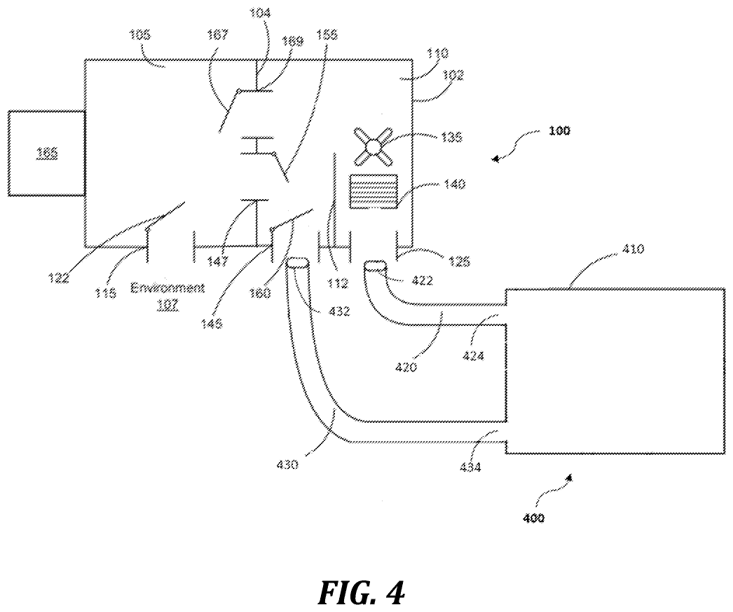

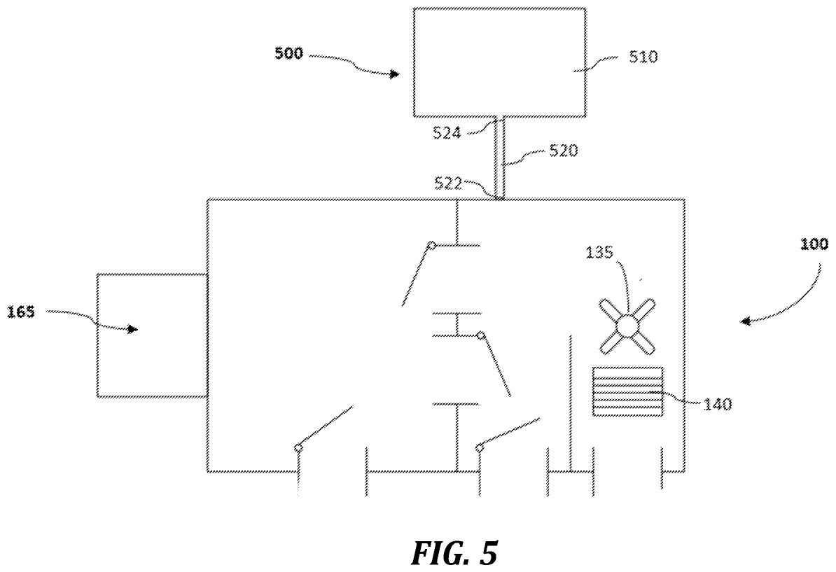

In some embodiments, the recirculating warm air system 100 comprises a cleaning unit 400 for decontaminating and/or otherwise cleaning the recirculating warm air machine 102. The cleaning unit 400 may be detachable, as illustrated in the example embodiment of FIG. 4, or it may be permanently connected to the recirculating warm air machine 102, as illustrated in the example embodiment of FIG. 5. The cleaning unit 400 may comprise various functionalities for removing contaminants from the warm air recirculating machine 102, although in the illustrated embodiment the cleaning unit 400 comprises an ionizer 410. Non-limiting examples of other cleaning units may include HEPA filters, UV light, activated carbon filters, and the like. The ionizer 410 may be configured to release ions into the warm air recirculating machine 102, which may attach themselves to contaminants within the warm air recirculating machine 102. In some embodiments, the ion-attached contaminants may then be deposited on collector plates within the ionizer 410. In some embodiments, air may be configured to pass into and out of the ionizer 410 via two different hoses. In other embodiments, air may be configured to pass out of the ionizer 410 via a single hose.

Where the cleaning unit 400 is detachably connected to the warm air recirculating machine 102, the cleaning unit 400 may comprise an intake hose 420 and an outlet hose 430. The ionizer 410 may connect to the warm air recirculating machine 102 using the intake hose 420 and the outlet hose 430. Specifically, the intake hose 420 may be configured to connect to the air outlet 125 by a first end 422 and may be configured to connect to the ionizer 410 via a second end 424 of the intake hose 420. When connected, the intake hose 420 may pass contaminated air from the recirculating warm air machine 102 to the ionizer 410. As the air passes through the ionizer 410, filters within the ionizer 410 may capture charged contaminants from the air. The outlet hose 430 may be configured to connect to at least one of the first air inlet 115 and/or the second air inlet 145. Specifically, the outlet hose 430 may be configured to connect to the first and/or second air inlets 115, 145 at a first end 432 and may be configured to connect to the ionizer 410 via a second end 434 of the outlet hose 430. When connected, the outlet hose 430 may pass cleansed air and/or air containing ions from the ionizer 410 to the recirculating warm air machine 102.

Where the cleaning unit 500 is permanently connected to the recirculating warm air machine 102, the cleaning unit 500 may comprise only a single hose 520. An ionizer 510 may connect to the recirculating warm air machine 102 using the hose 520. Specifically, the hose 520 may be configured to connect to an inlet formed within the air recirculating machine 102 at a first end 522 of the hose 520 and may be configured to connect to the ionizer 510 via a second end 524 of the hose 520. When connected, the hose 520 may pass ions from the ionizer into the recirculating warm air machine 102, which itself may recirculate the air within the machine 102 using a “cleaning” mode, e.g., via activation of the air blower 135.

In the illustrated embodiments, the ionizer 410, 510 is in operative communication with the second chamber 110 and in operative communication with both the first chamber 105 and the second chamber 110 while the fourth valve 167 and the second valve 155 are in the open position. However, it will be appreciated that the ionizer 410, 510 may be utilized to cleanse any portion of the warm air recirculating machine 102, as well as the entirety of the warm air recirculating machine 102. It will also be appreciated that the cleaning unit 400, 500 may be used to clean the air inflatable product 120.

The intake hose 420, the outlet hose 430, and/or the dual function hose 520 may be made of a flexible air-impermeable material, such as plastic or any other applicable material.

It will be appreciated that the cleaning units 400, 500 may be intended for use between patient uses of the recirculating warm air machine 102. In particular, where the cleaning unit 400 is detachable, the cleaning unit 400 may be configured to connect to the same inlets and/or outlets as the inflatable product 120. In such embodiments, the inflatable product 120 cannot be connected to the recirculating warm air machine 102 at the same time as the cleaning unit 400.

In still further embodiments, the processing unit 165 may be configured to determine that the intake hose 420 and/or the outlet hose 430 of the cleaning unit 400 are connected to the recirculating warm air machine 102. Upon determining that the intake hose 420 and/or the outlet hose 430 are connected to the recirculating warm air machine 102, the processing unit 165 may initiate the cleaning process by turning on the ionizer 410. Additionally or alternatively, the cleaning unit 400 may include a switch, button, or other control (not shown) to manually initiate the cleaning process.

In some embodiments, the cleaning unit 400, 500 may comprise a timing functionality that enables the ionizer 410, 510 to run for a determined period of time after it is switched on, thus enabling a user to turn on the ionizer 410, 510 and walk away without having to worry about turning off the ionizer. In some embodiments, controls on the ionizer 410, 510 itself may enable a user to set the period of time for which the ionizer 410, 510 is to run before shutting off automatically. Additionally or alternatively, a user may input the period of time to the processing unit 165, which then terminates the cleaning functionality after the predetermined period of time has elapsed. Additionally or alternatively, the period of time may be determined dynamically, by the processing unit. As non-limiting examples, the processing unit 165 may determine the period of time based on one or more of: a contaminant level in air received at the ionizer 410,510, a contaminant level of air in the warm air recirculating machine 102, an amount of time elapsed since the last cleaning, an indication of contamination level input by a user, and/or any other factors related to cleanliness of the system 100 or portions thereof.

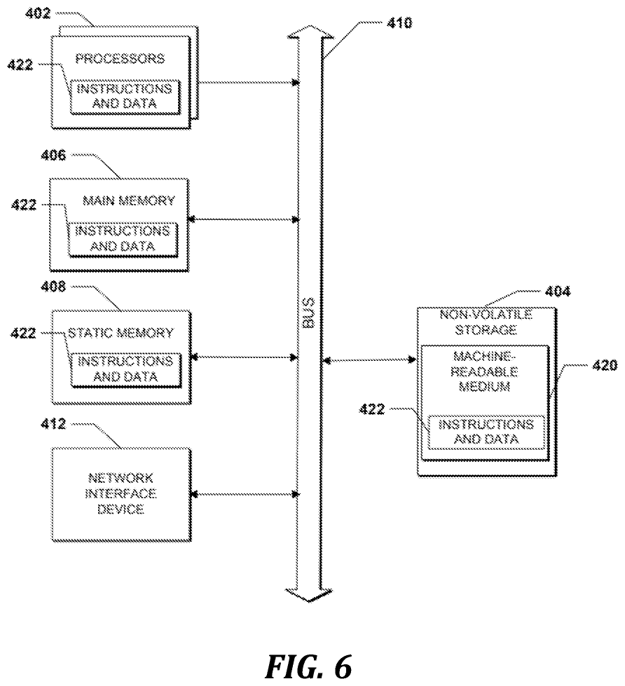

FIG. 6 shows a diagrammatic representation of a computing device for a machine in the exemplary electronic form of a computer system 400, within which a set of instructions for causing the machine to perform any one or more of the methodologies discussed herein can be executed. In various exemplary embodiments, the machine operates as a standalone device or can be connected (e.g., networked) to other machines. In a networked deployment, the machine can operate in the capacity of a server or a client machine in a server-client network environment, or as a peer machine in a peer-to-peer (or distributed) network environment. The machine can be a field programmable gate array, a personal computer (PC), a tablet PC, a set-top box, a cellular telephone, a digital camera, a portable music player (e.g., a portable hard drive audio device, such as an Moving Picture Experts Group Audio Layer 3 (MP3) player), a web appliance, a network router, a switch, a bridge, or any machine capable of executing a set of instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The computer system 400 may include a processor or multiple processors 402, a non-volatile storage 404, a main memory 406 and a static memory 408, which communicate with each other via a bus 410. The computer system 400 may also include a network interface device 412. The non-volatile storage 404 may include a machine-readable medium 420, which stores one or more sets of instructions and data 422 embodying or utilized by any one or more of the methodologies or functions described herein. The instructions and data 422 can also reside, completely or at least partially, within the main memory 406 and/or within the processors 402 during execution thereof by the computer system 400. The main memory 406 and the processors 402 also constitute machine-readable media.

While the machine-readable medium 420 is shown in an exemplary embodiment to be a single medium, the term “computer-readable medium” should be taken to include a single medium or multiple media (e.g., a centralized or distributed database, and/or associated caches and servers) that store the one or more sets of instructions. The term “computer-readable medium” shall also be taken to include any medium that is capable of storing, encoding, or carrying a set of instructions for execution by the machine and that causes the machine to perform any one or more of the methodologies of the present application, or that is capable of storing, encoding, or carrying data structures utilized by or associated with such a set of instructions. The term “computer-readable medium” shall accordingly be taken to include, but not be limited to, solid-state memories, optical and magnetic media. Such media can also include, without limitation, hard disks, floppy disks, NAND or NOR flash memory, digital video disks, Random Access Memory, Read-Only Memory, and the like.

Embodiments of the present disclosure provide a system operative by a set of methods comprising instructions configured to operate the aforementioned components in accordance with the methods. The following depicts an example of a method of a plurality of methods that may be performed by at least one of the aforementioned components. Various hardware components may be used at the various stages of operations disclosed with reference to each component.

For example, although methods may be described to be performed by a single component, it should be understood that, in some embodiments, different operations may be performed by different components in operative relation with one another. For example, an apparatus may be employed in the performance of some or all of the stages disclosed with regard to the methods. As such, the apparatus may comprise at least one architectural component disclosed herein.

Furthermore, although the stages of the following example method are disclosed in a particular order, it should be understood that the order is disclosed for illustrative purposes only. Stages may be combined, separated, reordered, and various intermediary stages may exist. Accordingly, it should be understood that the various stages, in various embodiments, may be performed in arrangements that differ from the ones claimed below. Moreover, various stages may be added or removed from the without altering or deterring from the fundamental scope of the depicted methods and systems disclosed herein.

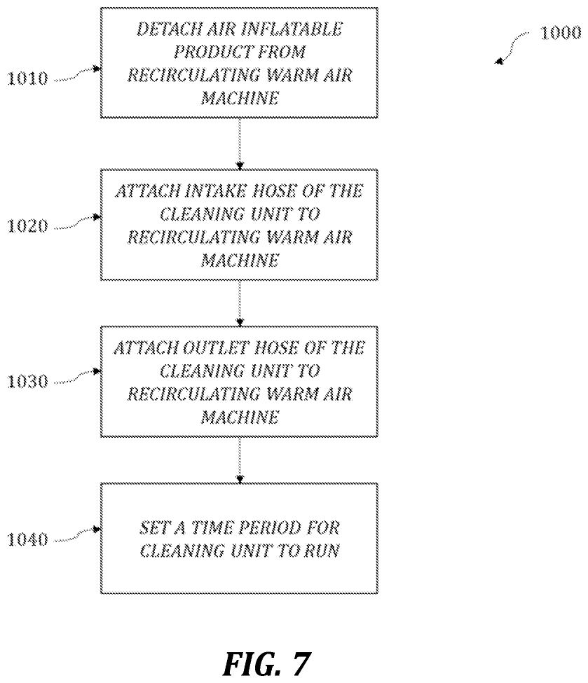

Consistent with embodiments of the present disclosure, a method may be performed by at least one of the aforementioned components. For example, FIG. 7 illustrates one example method 1000 of cleaning recirculating warm air systems as described herein. The method may begin at step 1010, which may involve detaching the air inflatable product 120 from the recirculating warm air machine 102. A subsequent step 1020 may involve attaching the intake hose 420 of the cleaning unit 400 to the air outlet 125 of the recirculating warm air machine 102. At step 1030, which may occur before, after, or simultaneously with step 1020, the method 1000 may involve attaching the outlet hose 430 to either the first air inlet 114 or the second air inlet 145 of the recirculating warm air machine 102. Where the cleaning unit 400 only requires one hose, the method 1000 only involves a single step of attaching a single hose to the recirculating warm air machine 102. Optionally, the method 1000 may further involve step 1040. Step 1040 may involve setting a time period for which the cleaning unit 400 should run.

The example embodiments described herein may be implemented in an operating environment comprising software installed on a computer, in hardware, or in a combination of software and hardware.

Thus, recirculating warm air systems have been described. Although embodiments have been described with reference to specific exemplary embodiments, it will be evident that various modifications and changes can be made to these exemplary embodiments without departing from the broader spirit and scope of the present application. Accordingly, the specification and drawings are to be regarded in an illustrative rather than a restrictive sense.

What is claimed is:

1. A recirculating warm air system, the system comprising:
an air inflatable product made of an air-impermeable material;
a recirculating warm air machine configured to supply air to the air inflatable product when removably attached thereto, the machine comprising:
a first chamber having a first air inlet configured to allow air to pass into the first chamber,
a second chamber having a second air inlet configured to allow air to pass into the second chamber and an air outlet configured to allow air to pass out of the second chamber, and
one or more one-way valves that are configured to selectively allow the passage of air between the first and second chambers to ensure re-circulation of heated air within the air inflatable product when the inflatable product is attached to the recirculating warm air machine; and
a detachable cleaning unit configured to removably attach to the recirculating warm air machine, the cleaning system comprising:
an ionizing unit,
an intake hose configured to connect to the air outlet of the recirculating warm air machine, and
an outlet hose configured to connect to either the first air inlet or the second air inlet of the recirculating warm air machine.

2. The recirculating warm air system of claim 1, wherein air is configured to pass from the second chamber of the air recirculating machine into the ionizing unit via the intake hose, through the ionizing unit, and back into the air recirculating machine via the outlet hose.

3. The recirculating warm air system of claim 1, wherein the recirculating warm air machine further comprises:
a first hose configured to connect to the air outlet and to the air inflatable product to pass the air from the air recirculating machine to the air inflatable product; and
a second hose configured to connect to the second air inlet and to the air inflatable product to receive the air passing from the air inflatable product.

4. The recirculating warm air system of claim 1, wherein the first and second air inlets have one-way valves located thereon, the one-way valves being configured to only allow air to flow into the recirculating warm air machine.

5. The recirculating warm air system of claim 4, wherein the air outlet has a one-way valve located thereon, the one-way valve being configured to only allow air to flow out of the recirculating warm air machine.

6. The recirculating warm air system of claim 1, wherein the recirculating warm air machine further comprises:
an air blower configured to move air toward the air outlet; and
a heater located between the air blower and the air outlet.

7. The recirculating warm air system of claim 6, further comprising a processing unit configured to control one or more of the following: the ionizer, the air blower, and the heater.

8. The recirculating warm air system of claim 7, wherein the processing unit is configured to control a time period for which the ionizer runs.

9. The recirculating warm air system of claim 8, wherein a user can select the time period for which the ionizer runs.

10. The recirculating warm air system of claim 9, wherein the processing unit is configured to determine that the ionizer is connected to the recirculating warm air machine and, upon determining that the ionizer is connected to the recirculating warm air machine, initiate cleaning of the recirculating air machine with the ionizer.

11. The recirculating warm air system of claim 7, wherein the processing unit is configured to determine that the inflatable air product is connected to the recirculating warm air machine; and
wherein a valve located on the first air inlet is opened upon the determining that the inflatable air product is connected to the recirculating warm air machine.

12. The recirculating warm air system of claim 11, wherein the processing unit is further configured to determine, based on predetermined criteria, that an amount of the air provided to the air inflatable product has reached a predetermined threshold; and
based on the determination:
close a second valve located between the first air chamber and the second air chamber; and
open a third valve located at the second air inlet;
wherein upon the closing of the second valve and the opening of the third valve, the air recirculates through the air inflatable product and the second chamber.

13. A recirculating warm air system, the system comprising:
a recirculating warm air machine configured to supply air to an air inflatable product when removably attached thereto, the machine comprising:
a first chamber having a first air inlet configured to allow air to pass into the first chamber,
a second chamber having a second air inlet configured to allow air to pass into the second chamber and an air outlet configured to allow air to pass out of the second chamber, and
one or more one-way valves that are configured to selectively allow the passage of air between the first and second chambers to ensure re-circulation of heated air within the air inflatable product when the inflatable product is attached to the recirculating warm air machine; and
a cleaning unit permanently attached to the recirculating warm air machine, the cleaning unit comprising:
an ionizing unit, and
a hose in operative communication with the second chamber, the hose being configured to deliver negative ions into the second chamber.

14. The recirculating warm air system of claim 13, wherein the recirculating warm air machine further comprises:
an air blower configured to move air toward the air outlet; and
a heater located between the air blower and the air outlet.

15. The recirculating warm air system of claim 14, further comprising a processing unit configured to control one or more of the following: the ionizer, the air blower, and the heater.

16. The recirculating warm air system of claim 15, wherein the processing unit is configured to control a time period for which the ionizer runs.

17. The recirculating warm air system of claim 16, wherein a user can select the time period for which the ionizer runs.

18. The recirculating warm air system of claim 15, wherein the processing unit is configured to determine that the air inflatable product is connected to the recirculating warm air machine.

19. A method of cleaning the recirculating warm air system of claim 1, the method comprising:
attaching the intake hose of the cleaning unit to the air outlet of the recirculating warm air machine;
attaching the outlet hose of the cleaning unit to either the first air inlet or the second air inlet of the recirculating warm air machine; and
setting a time period for which the cleaning unit should run.

20. The method of claim 19, further comprising, prior to attaching the intake and outlet hoses of the cleaning unit, detaching the air inflatable product from the recirculating warm air machine.
